(19) United States
(12) Patent Application Publication
(10) Pub. No.: US 2020/0343590 A1
(43) Pub. Date: Oct. 29, 2020

(54) NON-AQUEOUS ELECTROLYTE LIQUID FOR MAGNESIUM SECONDARY BATTERY AND MAGNESIUM SECONDARY BATTERY USING SAME

(71) Applicant: Panasonic Intellectual Property Management Co., Ltd., Osaka (JP)

(72) Inventors: TOMOFUMI HAMAMURA, Osaka (JP); TOSHIRO KUME, Hyogo (JP); KENSUKE NAKURA, Osaka (JP)

(21) Appl. No.: 16/924,134

(22) Filed: Jul. 8, 2020

Related U.S. Application Data
(63) Continuation of application No. PCT/JP2019/026873, filed on Jul. 5, 2019.

(30) Foreign Application Priority Data
Dec. 5, 2018 (JP) 2018-228378

(51) Int. Cl. H01M 10/0567; H01M 10/054; H01M 10/0568; H01M 10/0569; C07D 213/16

(52) U.S. Cl. CPC H01M 10/0567; H01M 10/054; H01M 2300/0025; H01M 10/0569; C07D 213/16; H01M 10/0568

(57) ABSTRACT

The non-aqueous electrolyte liquid for a magnesium secondary battery according to one aspect of the present disclosure contains a non-aqueous solvent, a magnesium salt, and an aromatic heterocyclic compound which has an aliphatic hydrocarbon group which is a substituent. The aromatic heterocyclic compound includes at least one selected from the group consisting of a nitrogen atom, an oxygen atom, a phosphorus atom, and a sulfur atom as a constituent atom of the ring thereof. The aromatic heterocyclic compound is a non-electrolyte.

### BACKGROUND

### 1. Technical Field

The present disclosure relates to a non-aqueous electrolyte liquid for a magnesium secondary battery and a magnesium secondary battery using the same.

### 2. Description of the Related Art

Recently, development of a magnesium secondary battery has been expected. Patent Literature 1 discloses an electrolyte liquid containing Mg(CH₃CN)₆(PF₆)₂.

### CITATION LIST

### Patent Literature

Patent Literature 1: Japanese Patent Application Publication No. 2017-145197

### SUMMARY

The present disclosure provides a novel non-aqueous electrolyte liquid for a magnesium secondary battery and a magnesium secondary battery using the same.

The present disclosure provides a non-aqueous electrolyte liquid for a magnesium secondary battery, the non-aqueous electrolyte liquid containing:

a non-aqueous solvent;

a magnesium salt; and

an aromatic heterocyclic compound which has an aliphatic hydrocarbon group which is a substituent,

wherein

the aromatic heterocyclic compound includes at least one selected from the group consisting of a nitrogen atom, an oxygen atom, a phosphorus atom, and a sulfur atom as a constituent atom of the ring thereof; and

the aromatic heterocyclic compound is a non-electrolyte.

A novel non-aqueous electrolyte liquid for a magnesium secondary battery and a magnesium secondary battery using the same are provided by the present disclosure.

### BRIEF DESCRIPTION OF THE DRAWINGS

FIG. 1 is a cross-sectional view showing a constructive example of a magnesium secondary battery schematically.

FIG. 2A is a graph showing a cyclic voltammogram of a sample 1 (sweep range: −1 V to 2 V).

FIG. 2B is a graph showing a cyclic voltammogram of the sample 1 (sweep range: 0 V to 2 V).

FIG. 2C is a graph showing a cyclic voltammogram of the sample 1 (sweep range: 0.5 V to 2 V).

FIG. 3 is a graph showing cyclic voltammograms of the sample 1 and a sample 2.

FIG. 4 is a graph showing cyclic voltammograms of the sample 1 and a sample 3.

### DETAILED DESCRIPTION OF THE EMBODIMENT

(Findings Which Established the Foundation of the Present Disclosure)

A magnesium secondary battery has been expected to be put to practical use as a secondary battery having high capacity, since two-electron reaction is used in the magnesium secondary battery. However, since interaction between divalent magnesium ions and a solvent therearound is strong, the solvent is less likely to be desorbed from the magnesium ions. In other words, deposition and dissolution of a magnesium metal is less likely to occur in an electrolyte liquid for the magnesium secondary battery. This is a problem unique to a non-aqueous electrolyte liquid for the magnesium secondary battery. For example, in an existing lithium ion battery, a non-aqueous electrolyte liquid provided by dissolving LiPF₆ in a solvent such as a carbonate is used. The deposition and dissolution of the magnesium metal do not occur in a non-aqueous electrolyte liquid provided by dissolving a magnesium salt such as Mg(AN)₆(PF₆)₂ in a carbonate (where AN means acetonitrile). Due to such problems, in the magnesium secondary battery, a strong limitation is imposed on the combination of a non-aqueous solvent and the magnesium salt.

To solve the problem, the present inventors found the following novel non-aqueous electrolyte liquid.

(Summary of One Aspect According to the Present Disclosure)

A non-aqueous electrolyte liquid for a magnesium secondary battery according to a first aspect of the present disclosure contains:

a non-aqueous solvent;

a magnesium salt; and

an aromatic heterocyclic compound which has an aliphatic hydrocarbon group which is a substituent,

wherein

the aromatic heterocyclic compound includes at least one selected from the group consisting of a nitrogen atom, an oxygen atom, a phosphorus atom, and a sulfur atom as a constituent atom of the ring thereof; and

the aromatic heterocyclic compound is a non-electrolyte.

In the first aspect, the aromatic heterocyclic compound having the aliphatic hydrocarbon group can form a coordination bond to magnesium ions competitively with a solvent, weaken interaction between the magnesium ions and the solvent, and facilitate the deposition and dissolution of the magnesium metal.

In a second aspect of the present disclosure, for example, in the non-aqueous electrolyte liquid for the magnesium secondary battery according to the first aspect, the aromatic heterocyclic compound may be represented by the following chemical formula (1):

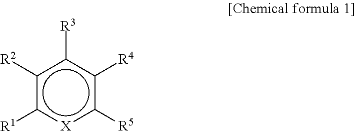

where

R¹ to R⁵ is, each independently, a hydrogen atom or an aliphatic hydrocarbon group;

at least one of R¹ to R⁵ is an aliphatic hydrocarbon group; and

X is a nitrogen atom or a phosphorus atom.

In a third aspect of the present disclosure, for example, in the non-aqueous electrolyte liquid for the magnesium secondary battery according to the second aspect, R³ may be an aliphatic hydrocarbon group. Coordination to Mg ions by a hetero atom included in an aromatic hetero ring (namely, X included in the above formula (1)) is facilitated more in a case where R³ is an aliphatic hydrocarbon group than in a case where any one of R¹, R², R⁴, and R⁵ is an aliphatic hydrocarbon group. This is because the coordination to the Mg ions by the hetero atom is less likely to be prevented, since R³ is the farthermost from the hetero atom included in the aromatic hetero ring. In the third aspect, R¹, R², R⁴, and R⁵ other than R³ is each independently, a hydrogen atom or an aliphatic hydrocarbon group.

In a fourth aspect of the present disclosure, for example, in the non-aqueous electrolyte liquid for the magnesium secondary battery according to the third aspect, R¹, R², R⁴, and R⁵ may be a hydrogen atom.

In a fifth aspect of the present disclosure, for example, in the non-aqueous electrolyte liquid for the magnesium secondary battery according to the first to fourth aspects, the aromatic heterocyclic compound may be an additive.

In a sixth aspect of the present disclosure, for example, in the non-aqueous electrolyte liquid for the magnesium secondary battery according to the first to fifth aspects, the aromatic heterocyclic compound may include a pyridine ring.

In a seventh aspect of the present disclosure, for example, in the non-aqueous electrolyte liquid for the magnesium secondary battery according to the first to sixth aspects, an anion included in the magnesium salt may be at least one selected from the group consisting of Cl⁻, Br⁻, I⁻, BF₄⁻, PF₆⁻, AsF₆⁻, SbF₆⁻, SiF₆⁻, ClO₄⁻, AlCl₄⁻, FSO₃⁻, CF₃SO₃⁻, C₄F₉SO₃⁻, [N(FSO₂)₂]⁻, [N(CF₃SO₂)₂]⁻, [N(C₂F₅SO₂)₂]⁻, [N(FSO₂)(CF₃SO₂)]⁻, CF₃BF₃⁻, C₂F₅BF₃⁻, and CB₁₁H₁₂⁻. These anions can form a salt with magnesium.

In an eighth aspect of the present disclosure, for example, in the non-aqueous electrolyte liquid for the magnesium secondary battery according to the seventh aspect, the anion included in the magnesium salt may be at least one selected from the group consisting of PF₆⁻, FSO₃⁻, [N(FSO₂)₂]⁻, [N(CF₃SO₂)₂]⁻, [N(C₂F₅SO₂)₂]⁻, and CB₁₁H₁₂⁻. A magnesium salt is formed with these anions to improve dissolubility.

In a ninth aspect of the present disclosure, for example, in the non-aqueous electrolyte liquid for the magnesium secondary battery according to the first to eighth aspects, the aliphatic hydrocarbon group may be a chain hydrocarbon group.

A magnesium secondary battery according to a tenth aspect of the present disclosure comprises:

a cathode;

an anode; and

a non-aqueous electrolyte liquid for a magnesium secondary battery according to any one of the first to ninth aspects.

In the tenth aspect, for example, the non-aqueous electrolyte liquid for the magnesium secondary battery according to any one of the first to ninth aspects is used to raise electrolytic chemical stability of the non-aqueous electrolyte liquid. As a result, the magnesium secondary battery fulfills its function.

Hereinafter, the non-aqueous electrolyte liquid according to the embodiment and the magnesium secondary battery using the same will be described with reference to the drawings.

The following descriptions all show general or specific examples. The numerical values, the composition, the shape, the film thickness, the electrical characteristics, and the structure of the secondary battery will be shown below are merely examples, and are not intended to limit the present disclosure. In addition, components that are not described in the independent claims that show the top-level concept are optional components.

(1. Non-aqueous Electrolyte Liquid)

The non-aqueous electrolyte liquid for the magnesium secondary battery according to the one aspect of the present disclosure contains a non-aqueous solvent, a magnesium salt, and an aromatic heterocyclic compound. The aromatic heterocyclic compound has an aliphatic hydrocarbon group which is a substituent. The magnesium salt and the aromatic heterocyclic compound are dissolved in the non-aqueous solvent.

The aromatic heterocyclic compound forms a coordination bond to magnesium ions competitively with a solvent, weakens the interaction between the magnesium ions and the solvent, and facilitates the deposition and dissolution of the magnesium metal. Therefore, the selectivity of the non-aqueous solvent can be expanded, depending on a desired condition. “The desired condition” may be, for example, at least one of that magnesium-ion conductivity is high, to be stable electrochemically, to be stable chemically, to be stable thermally, to be safe, that environmental burden is low, and to be inexpensive. For example, the magnesium salt is dissolved in the non-aqueous solvent at high concentration to raise the magnesium-ion conductivity of the non-aqueous electrolyte liquid. For example, a non-aqueous solvent having high oxidation resistance is selected to provide a non-aqueous electrolyte liquid having electrochemical stability. For example, a non-aqueous solvent having low toxicity is selected to provide a non-aqueous electrolyte liquid having high safety.

“Aromatic heterocyclic compound” in the present disclosure means a heterocyclic compound having aromaticity. The heterocyclic compound means a compound including at least one hetero atom as a constituent atom of the ring thereof. Examples of the hetero atom include a nitrogen atom, an oxygen atom, a phosphorus atom, and a sulfur atom. The aromatic heterocyclic compound is a non-electrolyte. In other words, the aromatic heterocyclic compound is not a salt. The aromatic heterocyclic compound is a substance which is unionized even if dissolved in a non-aqueous solvent.

The aromatic heterocyclic compound tends to exhibit a higher electron donation property than a carbonate. The coordination bond between the aromatic heterocyclic compound and the magnesium ions are formed more easily than coordination bond between the carbonate and the magnesium ions. In other words, the coordination bond between the aromatic heterocyclic compound and the magnesium ions are formed selectively, as compared to the coordination bond between the carbonate and the magnesium ions. As a result, the aromatic heterocyclic compound weakens the interaction between the magnesium ions and the solvent to allow the magnesium metal to be easily deposited and dissolved. The aromatic heterocyclic compound may include one or more hetero atoms having an unshared electron pair. The aromatic heterocyclic compound may include two or more hetero atoms.

Examples of the aromatic heterocyclic compound include a 2H-azirine derivative, an azeto derivative, a pyridine derivative, an imidazole derivative, a pyrazole derivative, an oxazole derivative, a thiazole derivative, an imidazoline derivative, a 2-oxirene derivative, an oxol derivative, an oxepin derivative, a thiirene derivative, a thiol derivative, and a thiepine derivative.

In the present embodiment, the aromatic heterocyclic compound having a pyridine ring may be used. The pyridine ring has a nitrogen atom having a high electron donation property. Also, the pyridine ring has high permittivity. Therefore, the coordination bond is selectively formed between the aromatic heterocyclic compound having a pyridine ring and the magnesium ions, and the aromatic heterocyclic compound having a pyridine ring is dissolved homogenously in the non-aqueous solvent.

The aromatic heterocyclic compound has an aliphatic hydrocarbon group which is a substituent. The aromatic heterocyclic compound may have a plurality of aliphatic hydrocarbon groups. The aliphatic hydrocarbon group may be a chain aliphatic hydrocarbon group. In more detail, the aliphatic hydrocarbon group may be linear or branched. Since the aromatic heterocyclic compound has an aliphatic hydrocarbon group, a three-dimensional volume of the aromatic heterocyclic compound is increased to improve the electrochemical stability of the aromatic heterocyclic compound kinetically. In this way, compatibility of the aromatic heterocyclic compound and a polar solvent is raised. The aliphatic hydrocarbon group may be connected directly to the hetero ring. The number of the carbon of the aliphatic hydrocarbon group is, for example, 1 to 4. Examples of the aliphatic hydrocarbon group include a methyl group, an ethyl group, an n-propyl group, an iso-propyl group, an n-butyl group, an iso-butyl group, a sec-butyl group, and a tert-butyl group. As an additive amount of the aromatic heterocyclic compound is increased, viscosity of an electrolyte liquid is increased. To prevent the viscosity of the electrolyte liquid from being increased, it is appropriate that the additive amount of the aromatic heterocyclic compound is not more than 50% at a volume ratio.

The magnesium salt has an anion. The anion is, for example, a monovalent anion.

The anion may be at least one selected from the group consisting of Cl⁻, Br⁻, I⁻, BF₄⁻, PF₆⁻, AsF₆⁻, SbF₆⁻, SiF₆⁻, ClO₄⁻, AlCl₄⁻, FSO₃⁻, CF₃SO₃⁻, C₄F₉SO₃⁻, [N(FSO₂)₂]⁻, [N(CF₃SO₂)₂]⁻, [N(C₂F₅SO₂)₂]⁻, [N(FSO₂)(CF₃SO₂)]⁻, CF₃BF₃⁻, C₂F₅BF₃⁻, and CB₁₁H₁₂⁻. The anion may be a derivative thereof. These anions may form a salt with magnesium.

From the viewpoint of the electrochemical stability, the anion may be at least one selected from the group consisting of BF₄⁻, PF₆⁻, ClO₄⁻, AlCl₄⁻, [N(CF₃SO₂)₂]⁻, [N(C₂F₅SO₂)₂]⁻, and CB₁₁H₁₂⁻.

From the viewpoint of the dissolubility, the anion may be at least one selected from the group consisting of PF₆⁻, FSO₃⁻, [N(FSO₂)₂]⁻, [N(CF₃SO₂)₂]⁻, [N(C₂F₅SO₂)₂]⁻, and CB₁₁H₁₂⁻. These anions raise the dissolubility of the magnesium salt in the solvent, and these anions raise ion dissociation of the dissolved magnesium salt.

The non-aqueous solvent is not particularly limited, as long as the non-aqueous solvent is a liquid capable of dissolving the magnesium salt. From the viewpoint of high permittivity, the non-aqueous solvent may include a cyclic carbonate, which raises the dissolubility of the magnesium salt in the non-aqueous solvent. An example of the cyclic carbonate is ethylene carbonate or propylene carbonate.

The non-aqueous solvent may contain other solvents. Examples of the other solvents include a cyclic ether, a linear ether, a borate ester, a cyclic sulfone, a linear sulfone, a nitrile, and a sultone.

(2. Magnesium Secondary Battery)

(2-1. Overall Structure)

The non-aqueous electrolyte liquid according to the present embodiment may be used for a magnesium secondary battery. The magnesium secondary battery comprises a cathode, an anode, and a non-aqueous electrolyte liquid having magnesium-ion conductivity. The non-aqueous electrolyte liquid described in the section “1. Non-aqueous electrolyte liquid” can be suitably used. The function of the magnesium secondary battery can be exhibited by using the non-aqueous electrolyte liquid of the present disclosure.

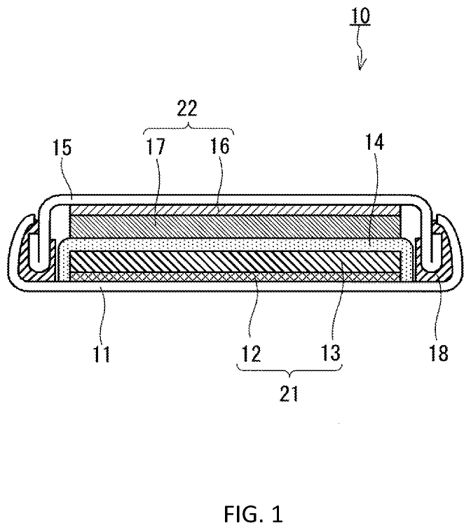

FIG. 1 is a cross-sectional view showing a constructive example of a magnesium secondary battery 10 schematically.

The magnesium secondary battery 10 comprises a cathode 21, an anode 22, a separator 14, a casing 11, a sealing plate 15, and a gasket 18. The separator 14 is disposed between the cathode 21 and the anode 22. The cathode 21, the anode 22, and the separator 14 are impregnated with the non-aqueous electrolyte liquid, and contained in the casing 11. The casing 11 is sealed with the gasket 18 and the sealing plate 15.

The structure of the magnesium secondary battery 10 may be, for example, a cylinder, a prism, a button, a coin, or a flat.

[2-2. Cathode]

The cathode 21 includes a cathode current collector 12 and a cathode active material layer 13 which is disposed on the cathode current collector 12. The cathode active material layer 13 is disposed between the cathode current collector 12 and the separator 14.

The cathode active material layer 13 contains a cathode active material. The cathode active material may be, for example, fluorinated graphite, a metal oxide, or a metal halide. The metal oxide and the metal halide may contain, for example, magnesium as well as at least one selected from scandium, titanium, vanadium, chromium, manganese, iron, cobalt, nickel, copper, and zinc. The cathode active material may be a sulfide such as Mo₆S₈ or a chalcogenide compound such as Mo₉Se₁₁.

Examples of the cathode active material include MgM₂O₄ (where M is at least one selected from Mn, Co, Cr, Ni and Fe), MgMO₂ (where M is at least one selected from Mn, Co, Cr, Ni and Al), MgMSiO₄ (where M is at least one selected from Mn, Co, Ni and Fe), and MgₓM_{y}AO_{z}F_{w} (where M is a transition metal, Sn, Sb or In; A is P, Si or S; 0<x≤2; 0.5≤y≤1.5; z is 3 or 4; and 0.5≤w≤1.5).

The cathode active material layer 13 may further contain a conductive material and/or a binder, if necessary.

Examples of the conductive material include a carbon material, a metal, an inorganic compound, and a conductive polymer. Examples of the carbon material include graphite, acetylene black, carbon black, ketjen black, carbon whisker, needle coke, and carbon fiber. Examples of the graphite include natural graphite and artificial graphite. Examples of the natural graphite include massive graphite and scale-shaped graphite. Examples of the metal include copper, nickel, aluminum, silver, and gold. Examples of the inorganic compound include tungsten carbide, titanium carbide, tantalum carbide, molybdenum carbide, titanium boride, and titanium nitride. These materials may be used alone or in combination of two or more.

Examples of the binder include fluorine-containing resin such as polytetrafluoroethylene (PTFE), polyvinylidene fluoride (PVdF), or fluororubber, thermoplastic resin such as polypropylene or polyethylene, ethylene propylene diene monomer (EPDM) rubber, sulfonated EPDM rubber, and natural butyl rubber (NBR). These materials may be used alone or in combination of two or more.

Examples of the solvent for dispersing the cathode active material, the conductive material, and the binder include N-methylpyrrolidone, dimethylformamide, dimethylacetamide, methyl ethyl ketone, cyclohexanone, methyl acetate, methyl acrylate, diethylenetriamine, N, N-dimethylaminopropyl amine, ethylene oxide, and tetrahydrofuran. For example, a viscosity increaser may be added to a dispersant. Examples of the viscosity increaser include carboxymethylcellulose and methylcellulose.

The cathode active material layer 13 is formed, for example, by the following method. First, the cathode active material, the conductive material, and the binder are mixed to provide a mixture thereof. Next, an appropriate solvent is added to the mixture to provide a cathode mixture paste. Next, the cathode mixture paste is applied to the surface of the cathode current collector 12, and then, dried. In this way, the cathode active material layer 13 is formed on the cathode current collector 12. The cathode active material layer 13 may be compressed to increase electrode density.

The film thickness of the cathode active material layer 13 is not particularly limited, and is, for example, not less than 1 μm and not more than 100 μm.

The material of the cathode current collector 12 is, for example, a metal or an alloy. More specifically, the material of the cathode current collector 12 is at least one kind of metal selected from the group consisting of copper, chromium, nickel, titanium, platinum, gold, aluminum, tungsten, iron, and molybdenum, or an alloy thereof. The material of the cathode current collector 12 may be, for example, stainless steel.

The cathode current collector 12 may be plate-shaped or foil-shaped. The cathode current collector 12 may be a stacking film.

If the casing 11 doubles as the cathode current collector, the cathode current collector 12 may be omitted.

[2-3. Anode]

The anode 22 includes, for example, an anode active material layer 17 containing an anode active material, and an anode current collector 16. The anode active material layer 17 is disposed between the anode current collector 16 and the separator 14.

The anode active material layer 17 contains an anode active material in and from which magnesium ions are allowed to be inserted during charge and desorbed during discharge, respectively. In this case, an example of the anode active material is a carbon material. Examples of the carbon material include graphite, non-graphite carbon, and a graphite intercalation compound. Examples of the non-graphitic carbon include hard carbon and coke.

The anode active material layer 17 may further contain a conductive material and/or a binder, if necessary. The conductive material, the binder, the solvent, and the viscosity increaser, each of which has been described in the section [2-2. Cathode], may be used appropriately for the anode active material layer 17.

The film thickness of the anode active material layer 17 is not particularly limited, and is, for example, not less than 1 μm and not more than 50 μm.

Alternatively, the anode active material layer 17 contains an anode active material on and from which magnesium is allowed to be deposited during the charge and dissolved into the non-aqueous electrolyte liquid during the discharge, respectively. In this case, examples of the anode active material include an Mg metal and an Mg alloy. The Mg alloy is, for example, an alloy of magnesium with at least one selected from aluminum, silicon, gallium, zinc, tin, manganese, bismuth, and antimony.

The same material as that of the cathode current collector 12 described in the section [2-2. Cathode] may also be used appropriately as the material of the anode current collector 16. The anode current collector 16 may be plate-shaped or foil-shaped.

If the sealing plate 15 doubles as the anode current collector, the anode current collector 16 may be omitted.

If the anode current collector 16 is composed of a material on and from which magnesium is allowed to be deposited during the charge and dissolved into the non-aqueous electrolyte liquid during the discharge, respectively, the anode active material layer 17 may be omitted. In other words, the anode 22 may be formed only of the anode current collector 16 on and from which magnesium is allowed to be deposited during the charge and dissolved into the non-aqueous electrolyte liquid during the discharge, respectively. In this case, the material of the anode current collector 16 may be stainless steel, nickel, copper, or iron.

[2-4. Separator]

Examples of the material of the separator 14 include a microporous thin film, a woven fabric, or a non-woven fabric. The material of the separator 14 may be a polyolefin such as polypropylene or polyethylene. The thickness of the separator 14 is, for example, 10 to 300 μm. The separator 14 may be a single layer film composed of one kind of a material, a composite film composed of two or more kinds of materials, or a multilayer film. The porosity of the separator 14 is, for example, within the range of 30 to 70%.

### EXAMPLES

(3. Experiment Results)

(3-1. Production of Non-aqueous Electrolyte Liquid)

(3-1-1. Sample 1)

First, a mixture solvent of ethylene carbonate and dimethyl carbonate (volume ratio 1:1) was prepared. As the aromatic heterocyclic compound, 4-tert-butylpyridine (CAS 3978-81-2, Mw: 135) was added to the provided mixture solvent so as to have a volume ratio of 1:1. In this way, a non-aqueous solvent was provided. Next, Mg(CH₃CN)₆(PF₆)₂, which was a magnesium salt, was dissolved in the non-aqueous solvent so as to have a concentration of 0.12 mol/L. In this way, a non-aqueous electrolyte liquid of the sample 1 was provided. The non-aqueous electrolyte liquid was prepared in an argon globe box. The 4-tert-butylpyridine was dehydrated overnight with a drying agent (trade name: Molecular Sieve (4A)) before the addition.

(3-1-2. Sample 2)

A non-aqueous electrolyte liquid was prepared in the same way as in the sample 1, except that 4-tert-butylpyridine was not added to the non-aqueous electrolyte liquid.

(3-1-3. Sample 3)

A non-aqueous electrolyte liquid was prepared in the same way as in the sample 1, except that pyridine was used in place of 4-tert-butylpyridine.

(3-2. CV Property Evaluation)

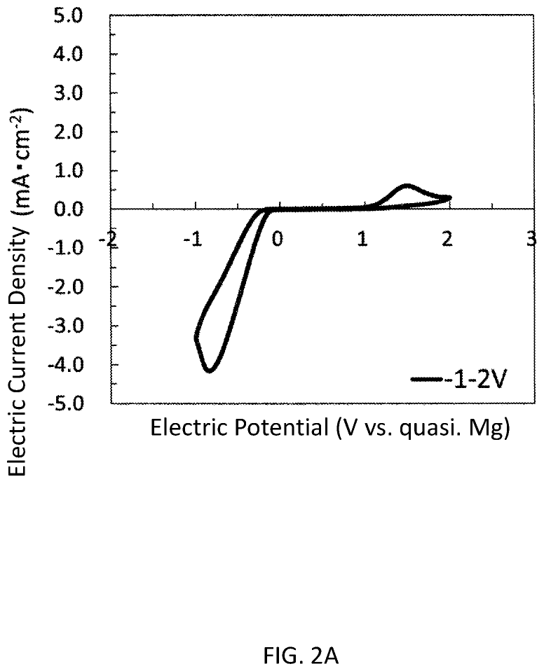

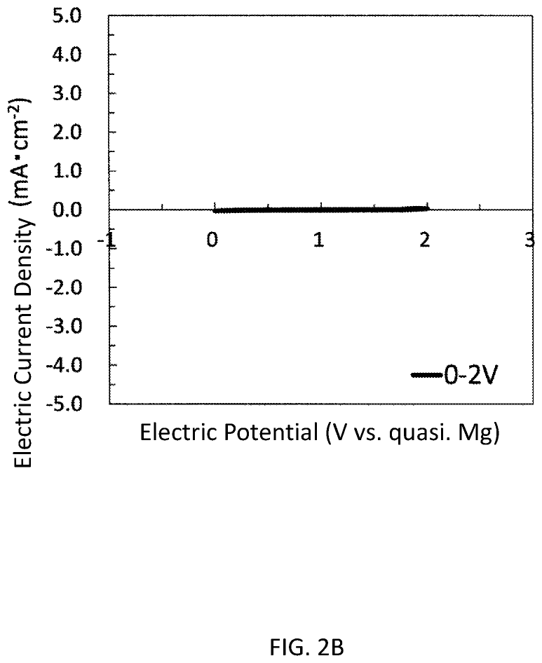

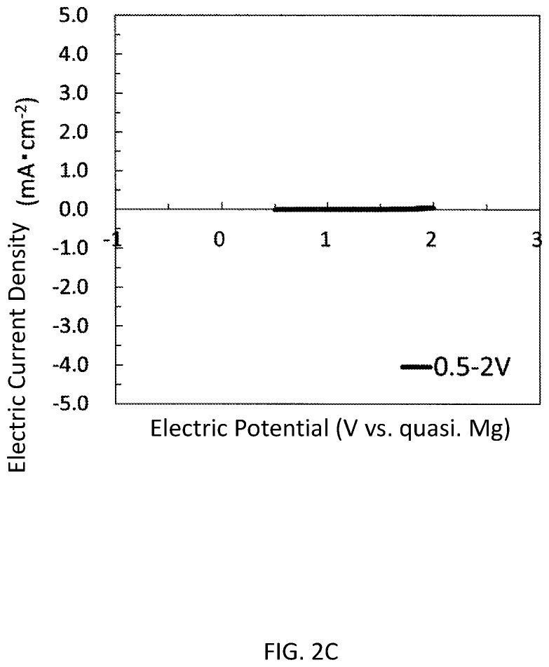

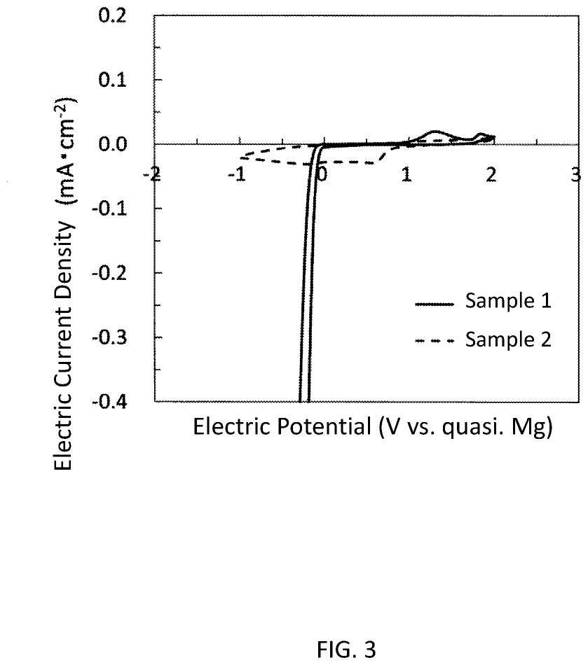

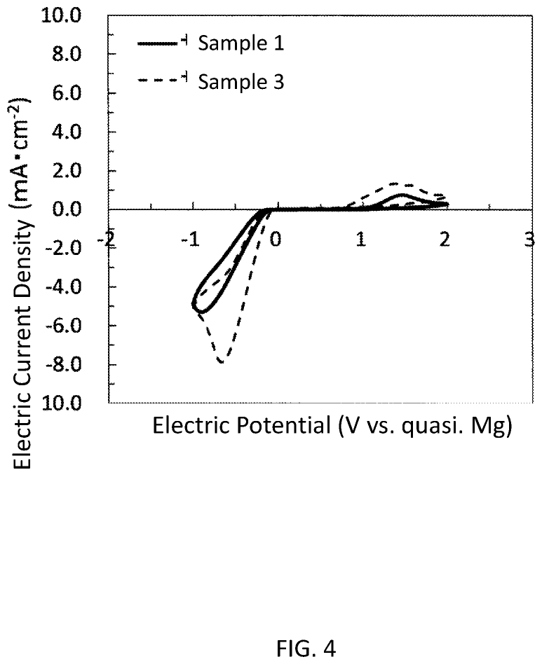

Each of the provided non-aqueous electrolyte liquids was subjected to a cyclic voltammetry (CV) measurement. A beaker cell was used as a measurement cell, and a potentio galvanostat (product of Bio-logic, VSP-300) was used as a measurement device. A sheet of aluminum foil of 5 mm×40 mm was used as a working electrode. Magnesium ribbons of 5 mm×40 mm each were used as a reference electrode and a counter electrode. The results are shown in FIG. 2A, FIG. 2B, FIG. 2C, FIG. 3, and FIG. 4.

FIGS. 2A to 2C are graphs each showing a cyclic voltammogram of the sample 1 The vertical axis represents an electric current which flowed through the working electrode, and the horizontal axis represents an electric potential of the working electrode with regard to the reference electrode. FIG. 2A shows the results within a sweep range of −1 to 2 V. FIG. 2B shows the results within a sweep range of 0 to 2 V. FIG. 2C shows the results within a sweep range of 0.5 to 2 V. A sweep rate of the electric potential was 5 mV/s. As shown in FIG. 2A, if the sweep range is −1 V to 2 V, namely, only if the electric potential was swept to a lower electric potential than an equilibrium electric potential of Mg/Mg²⁺, a correspondence oxidation electric current was observed. In other words, it is conceivable that the observed oxidation electric current is not a decomposition electric current of the solvent but an electric current which corresponds to a redox reaction. As shown in FIG. 2B and FIG. 2C, no electric current was observed, if the sweep range was 0 to 2 V or 0.5 to 2 V.

FIG. 3 is a graph showing cyclic voltammograms of the sample 1 and the sample 2. The sweep rate of the electric potential was 25 mV/s and the sweep range was −1.0 to 2.0 V. In the sample 1, reduction electric current and oxidation electric current were observed. In the sample 2, reduction electric current and oxidation electric current were hardly observed. In other words, it is conceivable that the reason why the reduction electric current and the oxidation electric current were observed in the sample 1 is that the 4-tert-butylpyridine contained in the sample 1 facilitates the deposition and the dissolution of the magnesium ions.

FIG. 4 is a graph showing cyclic voltammograms of the sample 1 and the sample 3. The sweep rate of the electric potential was 25 mV/s and the sweep range was −1.0 to 2.0 V. In both the sample 1 and the sample 3, the reduction electric current and the oxidation electric current were observed. The color of the non-aqueous electrolyte liquid of the sample 1 was not changed after the CV measurement. On the other hand, the color of the non-aqueous electrolyte liquid of the sample 3 was changed to be blue. It is conceivable that the reason why the color of the non-aqueous electrolyte liquid of the sample 1 was not changed is that the 4-tert-butylpyridine contained in the sample 1 raises the electrolytic chemical stability of the non-aqueous electrolyte liquid.

From the above results, it is conceivable that the non-aqueous electrolyte liquid of the sample 1 is appropriate for a magnesium secondary battery.

### INDUSTRIAL APPLICABILITY

The non-aqueous electrolyte liquid of the present disclosure can be used for a magnesium secondary battery.

### REFERENTIAL SIGNS LIST

10 Magnesium secondary battery
11 Casing
12 Cathode current collector
13 Cathode active material layer
14 Separator
15 Sealing plate
16 Anode current collector
17 Anode active material layer
18 Gasket
21 Cathode
22 Anode

1. A non-aqueous electrolyte liquid for a magnesium secondary battery, containing:
a non-aqueous solvent;
a magnesium salt;
an aromatic heterocyclic compound which has an aliphatic hydrocarbon group which is a substituent,
wherein
the aromatic heterocyclic compound includes at least one selected from the group consisting of a nitrogen atom, an oxygen atom, a phosphorus atom, and a sulfur atom as a constituent atom of the ring thereof; and
the aromatic heterocyclic compound is a non-electrolyte.

2. The non-aqueous electrolyte liquid for the magnesium secondary battery according to claim 1, wherein
the aromatic heterocyclic compound is represented by the following chemical formula (1): 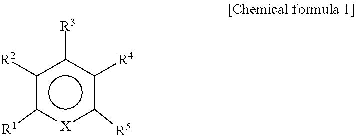
where
R¹ to R⁵ are, each independently, a hydrogen atom or an aliphatic hydrocarbon group;
at least one selected from the group consisting of R¹ to R⁵ is an aliphatic hydrocarbon group; and
X is a nitrogen atom or a phosphorus atom.

3. The non-aqueous electrolyte liquid for the magnesium secondary battery according to claim 2, wherein
R³ is an aliphatic hydrocarbon group.

4. The non-aqueous electrolyte liquid for the magnesium secondary battery according to claim 3, wherein
R¹, R², R⁴, and R⁵ are hydrogen atoms.

5. The non-aqueous electrolyte liquid for the magnesium secondary battery according to claim 1, wherein
the aromatic heterocyclic compound is an additive.

6. The non-aqueous electrolyte liquid for the magnesium secondary battery according to claim 1, wherein
the aromatic heterocyclic compound includes a pyridine ring.

7. The non-aqueous electrolyte liquid for the magnesium secondary battery according to claim 1, wherein
an anion included in the magnesium salt is at least one selected from the group consisting of Cl⁻, Br⁻, I⁻, BF₄⁻, PF₆^{-, AsF}₆⁻, SbF₆⁻, SiF₆^{-, ClO}₄⁻, AlCl₄⁻, FSO₃⁻, CF₃SO₃⁻, C₄F₉SO₃⁻, [N(FSO₂)₂]⁻, [N(CF₃SO₂)₂]⁻, [N(C₂F₅SO₂)₂]⁻, [N(FSO₂)(CF₃SO₂)]⁻, CF₃BF₃⁻, C₂F₅BF₃⁻, and CB₁₁H₁₂⁻.

8. The non-aqueous electrolyte liquid for the magnesium secondary battery according to claim 7, wherein
the anion included in the magnesium salt is at least one selected from the group consisting of PF₆⁻, FSO₃⁻, [N(FSO₂)₂]⁻, [N(CF₃SO₂)₂]⁻, [N(C₂F₅SO₂)₂]⁻, and CB₁₁H₁₂⁻.

9. The non-aqueous electrolyte liquid for a magnesium secondary battery according to claim 1, wherein
the aliphatic hydrocarbon group is a chain aliphatic hydrocarbon group.

10. The non-aqueous electrolyte liquid for a magnesium secondary battery according to claim 9, wherein
the aliphatic hydrocarbon group is a branched aliphatic hydrocarbon group.

11. The non-aqueous electrolyte liquid for a magnesium secondary battery according to claim 1, wherein
the aliphatic hydrocarbon group is an n-propyl group, an iso-propyl group, an n-butyl group, an iso-butyl group, a sec-butyl group, or a tert-butyl group.

12. The non-aqueous electrolyte liquid for a magnesium secondary battery according to claim 1, wherein
the non-aqueous solvent includes a cyclic carbonate, a cyclic ether, a linear ether, a borate ester, a cyclic sulfone, a linear sulfone, a nitrile, or a sultone.

13. A magnesium secondary battery, comprising:
a cathode;
an anode; and
the non-aqueous electrolyte liquid for the magnesium secondary battery according to claim 1.
